(19) United States
(12) Patent Application Publication
(10) Pub. No.: US 2003/0139946 A1
(43) Pub. Date: Jul. 24, 2003

(54) Method of collecting data on anxiety disorders and related research

(76) Inventors: Trevor David Vernon van Mierlo, Toronto (CA); Peter Gordon Farvolden, Toronto (CA); Jean-Paul Maurice Godmaire, Toronto (CA)

Correspondence Address: Piasetzki & Nenniger, Attn.: Gregory A. Piasetzki, 2308-120 Adelaide Street West, Toronto, ON, M5H 1T1, CA

(21) Appl. No.: 10/347,389

(22) Filed: Jan. 21, 2003

(30) Foreign Application Priority Data
Jan. 22, 2002 (CA) 2,368,992

(51) Int. Cl. G06F 17/60; A61B 5/00; G09B 3/00; G09B 7/00

(52) U.S. Cl. 705/2; 434/322; 600/300

(57) ABSTRACT

Diagnostic information relating to anxiety disorders is obtained by a method comprising the steps of (1) asking subjects questions relating to anxiety disorder symptoms; (2) receiving answers to the questions; (3) generating an original final report; (4) summarizing in the final report endorsed anxiety disorder symptoms; (5) indicating in the final report unendorsed anxiety disorder symptoms. Data relating to anxiety disorders is collected by keeping statistics on the answers given by the subjects and making the statistics available to researchers. Researchers are then provided access to the data from the population of subjects to conduct research on anxiety disorders. Also, the data collected is assessed to determine whether the questioning process should be modified in response to the data, so as to provide better information for researchers to use in their research.

### FIELD OF THE INVENTION

This invention relates to the field of data and information collection, and in particular, data and information collection relating to anxiety disorders.

### BACKGROUND OF THE INVENTION

In North America, anxiety disorders are among the most common of psychiatric conditions. According to one estimate, at any one time, as much as 10% of the population is afflicted with one or more anxiety disorders.

Anxiety disorders typically interfere significantly with the life of the sufferer. For example, this interference can take the form of compulsive behaviour, intense feelings of anxiety, fear or helplessness, or avoidance of situations which make the person anxious. Furthermore, individual anxiety disorders have high rates of co-morbidity with other psychiatric conditions, including other anxiety disorders, depression, and substance abuse. Thus, anxiety disorders take a substantial personal toll on those afflicted with them.

In additional to this personal toll, anxiety disorders are associated with large economic and social costs. According to one estimate, in the United States in 1990, costs associated with anxiety disorders totalled 46.6 billion U.S. dollars, or 31.5% of the total cost associated with mental illness. According to this same estimate, three-quarters of the costs associated with anxiety disorders were attributable to the reduced productivity of those affected by the disorder.

There are proven pharmacological and cognitive behavioural treatments for anxiety disorders. For example, there is good evidence for effective treatments for panic disorders (APA, 1998), specific phobias (Antony and Swinson, 1996), generalized anxiety disorder (Borovek and Costello, 1993; Goman and Kent, 1999), social phobia (Ballenger et al., 1998), obsessivecompulsive disorder (March et al., 1997) and post-traumatic stress disorder (Foa et al., 1999).

Some evidence suggests that first line treatment for anxiety disorders consists of behavioural therapy or cognitive behavioural therapy (CBT),sometimes in combination with selective serotonin reuptake inhibitor (SSRI) pharmacotherapy. Existing evidence suggests that these treatments work reasonably well when properly applied.

Unfortunately, many people do not seek appropriate treatment for anxiety disorders. For example, according to one estimate, in Ontario, Canada, only 20% of people with anxiety disorders seek treatment for their condition. By contrast, 86% of people with general health problems seek treatment from their primary care physicians. There are a number of possible reasons why such a relatively small number of anxiety disorder sufferers seek appropriate treatment. First, many people simply do not realize that anxiety disorders are conditions for which medical treatment would be appropriate. Unlike physical disorders, anxiety disorders are primarily emotional, and many people do not realize that difficult or extreme emotions (and related behaviour) might be proper subject matter for medical treatment, even if those emotions are interfering with living a normal life.

Second, patients with anxiety disorders often manifest physical problems, such as gastrointestinal problems, apparent heart problems or skin problems. However, they are often referred to physicians which specialize in treating those physical symptoms, rather than psychologists or psychiatrists capable of treating the underlying anxiety disorder. This is because the primary care physician who hears these complaints often does not associate the physical symptoms with the possibility of an anxiety disorder. For example, according to one estimate, as many as 80% of patients referred to a gastrointestinal clinic for investigation of possible Irritable Bowel Syndrome met the formal diagnostic criteria for a psychiatric disorder, primarily anxiety disorders (Lydiard, 1997).

Third, although behavioural therapy and CBT work reasonably well as treatments when they are used, these treatments are typically only available in larger urban centres. Furthermore, training for therapists in the administration of CBT is not widely available, and the administration of CBT requires considerable resources.

Anxiety disorders and depression are common mental health problems, and they are commonly co-morbid (i.e. they frequently occur together). Primary care physicians (typically general practitioners/family physicians) diagnose and treat the vast majority of people with these mental health problems, and may spend a major proportion of their working day doing so. According to some estimates, as much as 60% of a primary care physician's patients may present with some form of depressive or anxiety disorder, rather than a physical illness. Thus, primary care physicians, often pressed for time in managed care settings, are faced with the problem of accurately diagnosing a substantial number of people with depressive and anxiety disorders. This problem is magnified by the fact that diagnosing these types of disorders is not a skill that lies within the core speciality of most primary care physicians.

An accurate diagnosis of a depressive or anxiety disorder is labour-intensive. This is because arriving at an accurate diagnosis requires that the clinician consider potential alternative explanations for symptoms, as well as common co-morbid diagnoses. Thus, diagnosing mental health conditions requires substantial time and skill.

A number of structured and standardized assessment instruments have been developed to cover the major DSM-IV diagnoses. These include the Schedule for Affective Disorders (Endicott and Spitzer, 1978); the Anxiety Disorders Interview Schedule for DSM-IV (Brown, DiNardo and Barlow, 1994); the Mini-International Neuropsychiatric Interview (Sheehan et al., 1998); the Structured Clinical Interview for DSM-IV (S.C.I.D., First et al., 1995) and the Composite International Diagnostic Interview (Wittchen et al., 1994).

These structured assessment tools were first developed in academic centres. As a result, these assessment tools are structured according to the priorities of academics, not clinicians. For example, this type of tool was first used to standardize data collection in psychiatric epidemiology studies. Thus, these tools tend to be geared for collection of detailed and highly precise data. As a result, these interviews are often quite long and cumbersome to use. Also, because of their detailed and highly technical nature, these tools typically require that the person using them have extensive training, technical expertise and clinical experience in psychiatry or psychology. Thus, these interviews tend to be difficult and cumbersome to use.

However, as stated above, diagnosis of anxiety disorders most often takes place in the primary care setting, and it will be appreciated that the aforementioned tools are not well-suited to this setting. This is so for a number of reasons. First, primary care physicians typically see a relatively large volume of patients, and do not spend an extended period of time with any one patient. Thus, assessment tools or tests which take a long time to complete, and also take a long time for the diagnosing doctor to score, are unlikely to be used by primary care physicians with little time to spend with each patient. Second, though primary care physicians are most likely to be approached for diagnosis of an anxiety disorder, they are far less likely to have specialized training or clinical experience in psychology or psychiatry. Since such training and experience is often necessary for the proper use of these paper and pencil assessment tools, they are not well suited to a primary care setting.

With the shift in the delivery of the health care services that has resulted in primary care physicians becoming the primary providers of psychiatric treatment, there has been a growing need for brief self-report psychiatric screening instruments. There are now some paper-and-pencil screening instruments designed for use by primary care physicians, including the Symptom-Driven Diagnostic System, the Primary Care Evaluation of Mental Disorders and the M.I.N.I. Screen. These instruments are all 1-2 page screening instruments that are designed to be used in a primary care physician's waiting room.

There are also a number of computerized screening instruments available. Some are programs that guide clinicians through the diagnostic process for certain specific anxiety disorders. Others are designed to be self-administered by the patients. These include the SCID Screen Patient Questionnaire Computer Program and the SCID Screen Patient Questionnaire-Extended Computer Program. Also, on the Internet, there are currently a number of sites on which one can find disorder-specific “screeners” for various disorders, including panic disorder and depression.

Unfortunately, there are a number of problems with the available paper-and-pencil, Internet and computer-based screeners. First, they are often very narrow in scope. For example, there are a number of available paper-and-pencil and Internet screening instruments are available for individual anxiety disorders separately, and for major depression separately. A problem with such specific instruments is that they do not provide the broader screen of DSM-IV disorders that primary care clinicians need when they are trying to make an accurate diagnosis. If a doctor has only the information that a patient may have, for example, panic disorder, there are many important standard questions that the doctor must still ask to make a complete diagnosis. For example, to make an appropriate diagnosis, a doctor would need information about whether a patient has a common co-morbid condition, such as depression or another anxiety disorder. Thus, such screeners actually save very little time for the primary care physician trying to make a diagnosis, because he must still ask many more questions.

A related problem is that the screeners provide very little tangible information. Usually, they produce one of two possible outputs. If the patient has symptoms that indicate the possibility of a specific disorder, then the patient is simply informed that he may have the specific disorder and told to see a doctor. Otherwise, the patient is told that there is no evidence of a disorder. In either case, supporting information is often not provided. While some screeners do provide slightly more information, it is generally sparse. As a result, if the patient may have a disorder, the physician will have to ask the patient a series of detailed questions (similar to those answered by the patient in the original screener) to determine for himself the presence of symptoms and make a diagnosis. Thus, the patient may end up answering the same questions twice, while the doctor invests the same amount of time and effort as he would have even if the patient had not used the screener.

In addition, the existing brief, self-reporting paper and pencil instruments are, despite being shorter than the original lengthy paper and pencil instruments, somewhat impractical for use in primary care. Even these instruments still require someone to administer and score the tests. This is a considerable drain on the time and/or staff of a primary care physician, and it is therefore rarely done.

Another issue in the field of anxiety disorders is the organization of research activities and the manner in which those activities are undertaken. Specifically, research in the field of anxiety disorders tends to be highly competitive, with a wide variety of different organizations seeking funding on a competitive basis. This has a number of important results. First, the collection of data relating to anxiety disorders may often be done independently by different organizations, even when the data being collected by each organization are of similar scope, because the different organizations do not share their data at the time it is collected. Thus, the effort needed to collect data is often replicated by different organizations.

Second, because each of these organizations are competing with each other, each organization will tend to collect data for its own narrow research purposes only. As a result, these research organizations will tend to collect specific data rather than comprehensive and wide-ranging data on anxiety disorders.

Third, because of the competitive nature of the research, different organizations may not co-ordinate their research in a manner that might be advantageous from a scientific perspective. Because the organizations do not co-ordinate, they choose their research projects according to their own criteria or incentives, often without knowledge of similar decisions being made by other organizations. Thus, different organizations may choose independently to focus their research in similar areas, with the result that important research projects are delayed or not done at all.

### SUMMARY OF THE INVENTION

Therefore, what is desired is a method of obtaining diagnostic information that preferably facilitates diagnosis by a doctor. Also, preferably, a method is provided for keeping data, based on the diagnostic information obtained, for research. Also, there is preferably provided a method of keeping data on which diagnostic information is used by researchers, and which is not.

Therefore, according to one aspect of the invention, there is provided a method of obtaining diagnostic information relating to anxiety disorders, the method comprising the steps of:

(1) asking a subject questions relating to anxiety disorder symptoms, via computer;

(2) receiving answers to the questions;

(3) based on the answers, generating via the computer an original final report;

(4) summarizing in the final report endorsed anxiety disorder symptoms;

(5) indicating in the final report unendorsed anxiety disorder symptoms.

According to another aspect of the invention, there is provided a method of collecting data relating to anxiety disorders, the method comprising the steps of:

A) obtaining information from a population of subjects wherein each subject is questioned according to a subject interaction method comprising the steps of;

(1) questioning each subject via computer for anxiety disorder symptoms;

(2) receiving answers showing endorsed and unendorsed anxiety disorder symptoms;

B) keeping data comprising statistics on combinations of the answers given by the subject within the population, wherein it is possible to determine a number of subjects that gave particular combinations of answers.

C) making the data available to researchers.

According to another aspect of the invention, there is provided a method of collecting data relating to anxiety disorders, the method comprising the steps of:

A) obtaining information from a population of subjects by questioning each subject according to a subject interaction method comprising the steps of:

1) questioning each subject via computer for anxiety disorder symptoms and anxiety disorder subsymptoms;

2) receiving answers showing endorsed and unendorsed anxiety disorder symptoms and anxiety disorder subsymptoms;

B) keeping data comprising statistics on endorsed and unendorsed anxiety disorder symptoms within the population and endorsed and unendorsed anxiety disorder subsymptoms within the population.

C) making the data available to researchers.

According to another aspect of the invention, there is provided a method of collecting information on research being conducted, the method comprising:

(1) providing a collection of data units relating to one or more medical or psychological conditions;

(2) receiving requests for data units from one or more researchers;

(3) recording which data units are requested.

According to another aspect of the invention, there is provided a method of modifying a system for determining and reporting information relating to anxiety disorders, the method comprising:

(1) via a computer, questioning a population of subjects, according to a pre-existing questioning process, for information relating to anxiety disorders;

(2) via the computer, receiving answers from the subjects;

(3) determining from the answers if a previously specified data threshold has been reached;

(4) if the threshold has been reached, automatically and via the computer modifying the questioning process.

According to another aspect of the invention, there is provided a data threshold notification method, the method comprising the steps of:

1) via a computer, questioning a population of subjects for information relating to one or more medical or psychological conditions;

2) via the computer, receiving answers from the subjects;

3) determining from the answers if a previously specified data threshold had been reached;

4) if the threshold has been reached, automatically issuing an alert.

According to another aspect of the invention, there is provided a computer readable medium bearing instructions for realizing a method of obtaining diagnostic information relating to anxiety disorders, the instructions being arranged to cause one or more processors upon execution thereof to perform the following:

(1) asking a subject questions relating to anxiety disorder symptoms, via computer;

(2) receiving answers to the questions;

(3) based on the answers, generating via the computer an original final report;

(4) summarizing in the final report endorsed anxiety disorder symptoms;

(5) indicating in the final report unendorsed anxiety disorder symptoms.

According to another aspect of the invention, there is provided a computer readable medium bearing instructions for realizing a method of collecting data relating to anxiety disorders, the instructions being arranged to cause one or more processors upon execution thereof to perform the following:

A) obtaining information from a population of subjects wherein each subject is questioned according to a subject interaction method comprising the steps of:

(1) questioning each subject via computer for anxiety disorder symptoms;

(2) receiving answers showing endorsed and unendorsed anxiety disorder symptoms;

B) keeping data comprising statistics on combinations of the answers given by the subject within the population, wherein it is possible to determine a number of subjects that gave particular combinations of answers.

C) making the data available to researchers.

According to another aspect of the invention, there is provided a computer readable medium bearing instructions for realizing a method of collecting data relating to anxiety disorders, the instructions being arranged to cause one or more processors upon execution thereof to perform the following:

A) obtaining information from a population of subjects by questioning each subject according to a subject interaction method comprising the steps of:

1) questioning each subject via computer for anxiety disorder symptoms and anxiety disorder subsymptoms;

2) receiving answers showing endorsed and unendorsed anxiety disorder symptoms and anxiety disorder subsymptoms;

B) keeping data comprising statistics on endorsed and unendorsed anxiety disorder symptoms within the population and endorsed and unendorsed anxiety disorder subsymptoms within the population.

C) making the data available to researchers.

According to another aspect of the invention, there is provided a computer readable medium bearing instructions for realizing a method of collecting information on research being conducted, the instructions being arranged to cause one or more processors upon execution thereof to perform the following:

(1) providing a collection of data units relating to one or more medical or psychological conditions;

(2) receiving requests for data units from one or more researchers;

(3) recording which data units are requested.

According to another aspect of the invention, there is provided a computer readable medium bearing instructions for realizing a method of modifying a system for determining and reporting information relating to anxiety disorders, the instructions being arranged to cause one or more processors upon execution thereof to perform the following:

(1) via a computer, questioning a population of subjects, according to a pre-existing questioning process, for information relating to anxiety disorders;

(2) via the computer, receiving answers from the subjects;

(3) determining from the answers if a previously specified data threshold has been reached;

(4) if the threshold has been reached, automatically and via the computer modifying the questioning process.

According to another aspect of the invention, there is provided a computer readable medium bearing instructions for realizing a data threshold notification method, the instructions being arranged to cause one or more processors upon execution thereof to perform the following:

1) via a computer, questioning a population of subjects for information relating to one or more medical or psychological conditions;

2) via the computer, receiving answers from the subjects;

3) determining from the answers if a previously specified data threshold had been reached;

4) if the threshold has been reached, automatically issuing an alert.

### BRIEF DESCRIPTION OF THE DRAWINGS

FIG. 1 is a schematic drawing of the method of administration of the anxiety test according to the present invention;

FIG. 2 is a sample final report according to the present invention;

FIG. 3 is a sample data retrieval interface according to the present invention;

FIG. 4 is a schematic drawing of the researcher data retrieval process according to the present invention;

FIG. 5 is a chart showing the primary functions of the software engine according to the present invention.

### DETAILED DESCRIPTION OF THE PREFERRED EMBODIMENTS

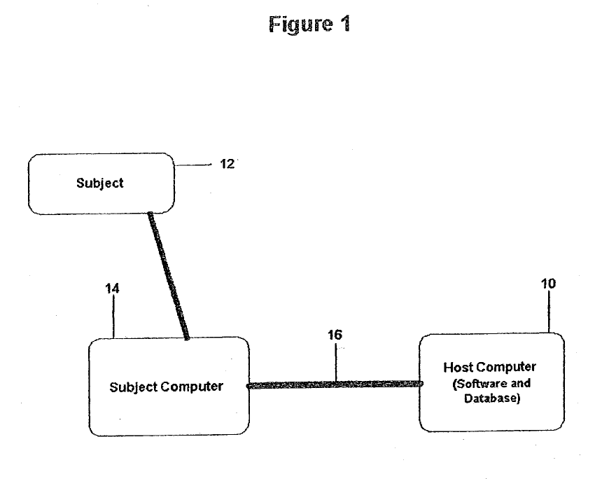

According to one aspect of the present invention, there is preferably provided a method of obtaining diagnostic information relating to anxiety disorders in patients. Preferably, as shown in FIG. 1, the method is carried out via a software-operated Internet website operating on a host computer 10. As will be more particularly described below, a subject 12 logging on to the website via a subject computer 14 and an Internet connection 16 is provided with an anxiety test which questions the subject about the presence of symptoms of one or more anxiety disorders. Preferably, the subject is also questioned for the presence of symptoms of common co-morbid conditions (i.e. conditions that have a significant positive correlation with one or more of the anxiety disorders). After the subject has completed the anxiety test, a final report is generated which preferably reports (a) the possibility of an anxiety disorder if one or more symptoms have been indicated by the subject, and (b) a summary of the symptoms that have been indicated. This report can then be printed out and taken by the subject to his doctor, or e-mailed directly to his doctor. A button is provided which, when selected, causes an e-mail message to appear, thus facilitating the e-mailing of the report to a doctor by the subject. A printer-friendly version of the final report can also be selected to facilitate printing.

It will be appreciated that the use of the Internet as a medium for the anxiety test is preferred for a number of reasons. First, the sufferers of anxiety disorders might be highly sensitive to the possibility that their conditions, including the accompanying (and likely irrational) fears, anxieties and behaviours, will be discovered. Second, such a person may be unwilling to meet face-to-face with a psychologist, psychiatrist or other doctor to undergo a detailed screening for anxiety disorder. This is because many people suffering from anxiety disorders may be having feelings or engaging in behaviours that they consider embarrassing, and therefore do not want to discuss with anyone.

It will be appreciated that providing initial anxiety disorder testing over the Internet may be helpful to a subject with these concerns, because the subject can undergo the testing process in the privacy of his own home. Thus, he can remain anonymous and not fear that sensitive or embarrassing information about him will be discovered. Also, because he is doing the anxiety test alone in his own home, he is not required, at least initially, to talk face-to-face with another person about feelings and behaviours that may embarrass him.

Preferably, the anxiety test will test the subject not only for the presence or absence of a single anxiety disorder, but rather, for symptoms of a number of anxiety disorders and common co-morbid conditions. It will be appreciated that individual anxiety disorders are often conditions that are commonly common co-morbid with other anxiety disorders. Also, other conditions, such as, for example, depression and substance abuse, are also often common comorbid conditions.

In the preferred embodiment, the subject is presented with a pre-screen, containing a set of questions, for determining whether the subject may have any symptoms of a pre-determined set of anxiety disorders and common co-morbid conditions. Most preferably, the set of disorders and conditions will include: panic disorder, obsessive-compulsive disorder (“OCD”), social phobia, generalized anxiety disorder (“GAD”), post-traumatic stress disorder (“PTSD”), specific phobias, agoraphobia and major depression.

It will be appreciated that panic disorder, OCD, social phobia, GAD, PTSD and specific phobias are anxiety disorders, which often occur together with one another, i.e. they are often commonly co-morbid. Agoraphobia is also a common co-morbid condition often associated with panic disorder. Similarly, major depression is commonly co-morbid with all of the anxiety disorders.

It will also be appreciated that, though substance abuse is commonly comorbid with various anxiety disorders, it is preferable not to question subjects about substance abuse. The reason is that, since substance abuse often involves illegal conduct, a subject may be unwilling to answer questions about it, particularly when the anxiety test is being taken over the Internet. The subject may fear that the information may fall into the hands of law enforcement. Thus, if questions regarding substance abuse are asked, subjects may be less willing to take the test. Since it is preferable that the test facilitate the diagnosis and treatment of anxiety disorders, it is preferable not to ask questions about the substance abuse, since such questions may dissuade the subject from taking the test. Rather, it is believed that it is most appropriate that questions relating to substance abuse be posed by the physician who makes the ultimate diagnosis of the patient. Because substance abuse is commonly co-morbid with anxiety disorders, if the consulting physician diagnoses an anxiety disorder, he will likely understand that there is a likelihood of substance abuse. Furthermore, physicians are bound by ethical duties of confidentiality, and in some jurisdictions, communications between doctor and patient are privileged. Thus, while a patient may be unlikely to disclose his substance abuse over the Internet, his is more likely to feel comfortable disclosing it to his doctor.

Based on the responses of the subject to the questions on the prescreen, the subject is presented with detailed screens, each containing a set of questions relating to specific anxiety disorders or co-morbid conditions to determine the presence or absence of specific symptoms. Thus, for example, if the subject's answers to the pre-screen questions indicate the possible presence of symptoms of OCD, the subject will be presented with a detailed screen which questions the subject in greater detail for the presence of symptoms of OCD. By contrast, if, based on the subject's answers to the prescreen, no symptoms of OCD appear to be present, the OCD detailed screen will not be presented to the subject.

Thus, it will be appreciated by those skilled in the art that the use of a pre-screen has the overall effect of shortening the length of time needed to complete the anxiety test. This is because the pre-screen is used to determine which detailed screens should be presented to the subject. Thus, the subject is only required to answer detailed questions for disorders that the pre-screen shows he might have. The subject is not required to answer detailed questions about any disorders whose possible presence is not indicated by the prescreen. This, in turn, makes the testing process more attractive to subjects, because it shortens the testing process and saves the subject the effort of answering a significant number of inappropriate questions. Therefore, it will be appreciated that the pre-screen questions are carefully designed to determine whether the subject may have symptoms of various disorders, while filtering out, in respect of each disorder, those subjects who clearly do not have sufficient symptoms to warrant further screening.

In the preferred embodiment, there are eight detailed screens available for presentation to the subject, depending on the answers given to the questions in the pre-screen. These are Screen A—Panic Disorder Screen; Screen B—Agoraphobia Screen; Screen C—Panic and Agoraphobia Screen; Screen D—Social Phobia Screen; Screen E—OCD Screen; Screen F—GAD Screen; Screen G—PTSD Screen; and Screen H—Depression Disorder Screen.

It will be appreciated that, in practice, many subjects will be questioned using more than one of these detailed screens, based on their answers to the pre-screen questions. This is because, as stated above, many of the conditions being assessed in the detailed screens are commonly co-morbid with other conditions questioned for in the detailed screens. Furthermore, even if not commonly co-morbid, some subjects may give answers to questions in the prescreen suggesting symptoms of more than one disorder or condition.

In the preferred embodiment, the pre-screen will include the following questions. The subject is asked to answer “yes” or “no” to each one. In the pre-screen, as well as the detailed screens described below, a symptom or question is endorsed if the subject answers “yes” and unendorsed if the subject answers “no”. It will be appreciated that other methods of endorsing symptoms could have been used. What is important is that the subject be provided with a way to indicate which symptoms are present and which are absent.

1. Have you ever had a sudden period of intense fear, anxiety, or discomfort (anxiety attack)?

2. Are you anxious about going to or being in some places or situations because you:

fear you will have an anxiety attack?

fear you will not be able to escape if you have an anxiety attack?

fear that help will not be there if you need it?

feel uncomfortable?

3. Do you avoid going to or being in some places or situations because you:

fear you will have an anxiety attack?

fear you will not be able to escape if you have an anxiety attack?

fear that help will not be there if you need it?feel uncomfortable?

4. Do you have an excessive fear of, or do you avoid social or work situations because you feel embarrassed, humiliated, or feel that people are judging you?

5. Do you experience anxiety because of uncontrollable thoughts, images, or impulses that you can't control?

6. Do you do certain things or repeat certain thoughts over-and over again?Do you do these things according to special rules, or until is feels just right? (for example: washing, ordering, checking, praying, counting, or repeating words)

7. For the past six months or more have you been worrying constantly or excessively about several different things? (for example, work, school, family, finances, or health)

8. Have you experienced or seen a traumatic or terrible event that included death or serious harm, or the threat of death or serious harm, to you or someone else? (for example: sexual assault, rape, accident, assault, disaster, war, or torture)

9. For the past two weeks or more have you been feeling depressed, sad, or flat for most of the time?

10. For the past two weeks or more have you lost interest or pleasure in things you usually like?

11. For the past two years or more have you felt depressed, sad, or flat for most of the time?

It will be appreciated by those skilled in the art that if the subject answers “yes” to one or more of these questions, he will be presented with the relevant detailed screen, depending on his answers or combination of answers. For example, if the subject answers “yes” to question 1, (but not to question 2 and not to question 3), he will be presented with Screen A—Panic Disorder Screen, because question 1 relates to symptoms of panic disorder. If the subject answers “yes” to one of question 2 or 3 (but not question 1) he will be presented with Screen B—Agoraphobia Screen, as questions 2 and 3 relate to symptoms of agoraphobia. If the subject answers “yes” to question 1 and to one or both of questions 2 or 3, he will be presented with Screen C—Panic and Agoraphobia Screen.

If the subject answers “yes” to question 4, then he is presented with Screen D—Social Phobia Screen, as question 4 relates to symptoms of social phobia. If the subject answers “yes” to one or both of questions 5 and 6, he will be presented with Screen E—OCD Screen, as questions 5 and 6 relate to symptoms of OCD. If the subject answers “yes” to question 7, then the subject will be presented with Screen F—GAD Screen, as question 7 relates to symptoms relating to GAD. If the subject answers “yes” to question 8, then he will be presented with Screen G—PTSD Screen, as question 8 relates to the possibility of symptoms of PTSD.

If the subject answers “yes” to one, two or all three of questions 9 to 11, then the subject is presented with Screen G—Depression Disorder Screen, as these questions relate to symptoms of depression.

If the subject does not answer “yes” to any of the questions, then no detailed screens are presented. Preferably, in such a case, a default final report is generated. The default final report preferably informs the subject that the concerns that the subject has regarding his or her health may not be the result of an anxiety problem, but that the anxiety test is not a substitute for a doctor's advice, and is not a diagnosis. Thus, the subject is preferably informed that, if he has concerns about his health, he should see his doctor.

In the preferred embodiment, the following introductory text is included in Screen A—Panic Disorder Screen: “According to your previous responses, you have had a sudden period of intense fear, anxiety, or discomfort (an anxiety attack). Please answer the following questions about your anxiety attack(s).” Also in the preferred embodiment, the following questions are included in Screen A—Panic Disorder Screen. The subject is asked to select “yes” or no, unless multiple subsymptom choices are given, in which case the subject is asked to select all answers that are applicable.

Did your first anxiety attack catch you by surprise, was it unexpected, or did it seem to “happen out of nowhere”?

Have you had more than one anxiety attack?

Does the worst part of the anxiety attack usually last between 10 and 30 minutes?

Have you ever spent a month or more worrying about having an anxiety attack or what might happen if you had another one?

Think back to your last bad anxiety attack and check the symptoms that you experienced:

skipping, racing, or pounding heart

sweaty or clammy hands

shortness of breath or trouble breathing

choking or lump in the throat

chest pain, pressure, or discomfort

nausea, diarrhea, or other stomach problems

dizziness, lightheadedness, or faintness

a feeling of being detached or outside of your body or a feeling that things around you were strange, weird, unreal, detached, or unfamiliar

thinking, feeling or fearing that you were losing control or going crazy

thinking, feeling, or fearing that you were dying

numbness or tingling in your body

chills or hot flushes

other (write in)

other (write in)

Have you had 6 or more anxiety attacks in the past year?

Have you had 2 or more anxiety attacks in the past two weeks?

How much do your anxiety attacks interfere with your normal daily life?(choose one)

no interference

mild/a little

moderate/medium interference

considerable/much interference

extreme/severe interference

In the preferred embodiment, Screen B—Agoraphobia Screen has three possible introductory paragraphs. The first possible introductory paragraph reads: “According to your previous responses, you are anxious about going to or being in places or situations because you fear having an anxiety attack, fear that you would not be able to escape if you had an anxiety attack, fear that help would not be there if you needed it, or because it makes you feel uncomfortable. Please answer the following questions about your anxiety.”

The second reads: “According to your previous responses, you avoid going to or being in places or situations because you fear having an anxiety attack, fear that you would not be able to escape if you had an anxiety attack, fear that help would not be there if you needed it, or because it makes you feel uncomfortable. Please answer the following questions about your avoidance.”

The third reads: “According to your previous responses, you are anxious and avoid going to or being in places or situations because you fear having an anxiety attack, fear that you would not be able to escape if you had an anxiety attack, fear that help would not be there if you needed it, or because it makes you feel uncomfortable. Please answer the following questions about your anxiety and avoidance.”

It will be appreciated that the introductory paragraphs (in this detailed screen, as well as the other detailed screens) serve to summarize for the subject the answers that he gave to the pre-screen questions, and to indicate the subject matter of the answers to be given in the detailed screen. Thus, in detailed screens such as Screen B—Agoraphobia Screen in which various combinations of pre-screen answers will lead to the detailed screen being presented, a plurality of different possible introductory paragraphs is provided so that, regardless of the subject's answers to the pre-screen questions, they will be accurately summarized. During the administration of a particular test to a particular subject, the single introductory paragraph that accurately summarizes the subject's previous answers is used.

In the preferred embodiment, the following questions will be presented in Screen B—Agoraphobia Screen. The subject is asked to answer “yes” or “no”. Where multiple subsymptom choices are given, the subject is asked-to select all applicable answers. In this screen, and in all others, when the subject selects a subsymptom, he endorses it as being present. Thus, all unselected subsymptoms are unendorsed.

Which situations do you fear and/or avoid?

Transportation

buses

trains

subways

streetcars

airplanes

riding in cars at any time

riding in cars on busy roads

driving a car at any time

driving a car on busy roads

Public Places

malls

stores

auditoriums or stadiums

theatres

grocery stores supermarkets

restaurants

classrooms

churches

museums

coffee shops

crowds

Enclosed Spaces

elevators

parking garages

bridges

tunnels

Open Spaces

open fields

wide streets

parks

exposed places

large rooms

lobbies

large open spaces

Being Alone

being at home alone

being away from home

standing in lines

Specific Situations

animals

heights

needles/blood

flying

thunderstorms

the dark

dentists

other (write-in)

other (write-in)

If you selected any of the above, how much does your fear or avoidance interfere with your normal daily life? (choose one)

no interference

mild/a little

moderate/medium interference

considerable/much interference

extreme/severe interference

In the preferred embodiment, Screen C—Panic Disorder and Agoraphobia Screen has three possible introductory paragraphs. The first possible introductory paragraph reads: “According to your previous responses, you have had a sudden period of intense fear, anxiety, or discomfort (anxiety attack). You have also reported anxiety in places or situations because you fear having an anxiety attack, fear that you would not be able to escape if you had an anxiety attack, fear that help would not be there if you needed it, or because it makes you feel uncomfortable. Please answer the following additional questions about your experience.”

The second reads: “According to your previous responses, you have had a sudden period of intense fear, anxiety, or discomfort (anxiety attack). You have also reported avoidance of places or situations because you fear having an anxiety attack, fear that you would not be able to escape if you had an anxiety attack, fear that help would not be there if you needed it, or because it makes you feel uncomfortable. Please answer the following additional questions about your experience.”

The third reads: “According to your previous responses, you have had a sudden period of intense fear, anxiety, or discomfort (anxiety attack). You have also reported fear and avoidance of places or situations because you fear having an anxiety attack, fear that you would not be able to escape if you had an anxiety attack, fear that help would not be there if you needed it, or because it makes you feel uncomfortable. Please answer the following additional questions about your experience.”

In the preferred embodiment, the following questions will be presented in Screen C—Panic Disorder and Agoraphobia Screen. The subject is asked to answer “yes” or “no”, unless multiple subsymptom choices are given, in which case the subject is asked to select all applicable answers.

Did your first anxiety attack catch you by surprise, was it unexpected, or did it seem to “happen out of nowhere”?

Have you had more than one anxiety attack?

Does the worst part of the anxiety attack usually last between 10 and 30 minutes?

Have you ever spent a month or more worrying about having an anxiety attack or what might happen if you had another one?

Think back to your last bad anxiety attack and check the symptoms that you experienced:

skipping, racing, or pounding heart

sweaty or clammy hands

shortness of breath or trouble breathing

choking or lump in the throat

chest pain, pressure, or discomfort

nausea, diarrhea, or other stomach problems

dizziness, lightheadedness, or faintness

a feeling of being detached or outside of your body or a feeling that things around you were strange, weird, unreal, detached, or unfamiliar

thinking, feeling or fearing that you were losing control or going crazy

thinking, feeling, or fearing that you were dying

numbness or tingling in your body

chills or hot flushes

other (write in)

other (write in)

Have you had 6 or more anxiety attacks in the past year?

Have you had 2 or more anxiety attacks in the past two weeks?

How much do your anxiety attacks interfere with your normal daily life?(choose one)

no interference

mild/a little

moderate/medium interference

considerable/much interference

extreme/severe interference

Which situations do you avoid because of fear or discomfort?

Transportation

buses

trains

subways

streetcars

airplanes

riding in cars at any time

riding in cars on busy roads

driving a car at any time

driving a car on busy roads

Public Places

malls

stores

auditoriums or stadiums

theatres

grocery stores/supermarkets

restaurants

classrooms

churches

museums

coffee shops

crowds

Enclosed Spaces

elevators

parking garages

bridges

tunnels

Open Spaces

open fields

wide streets

parks

exposed places

large rooms

lobbies

large open spaces

Being Alone

being at home alone

being away from home

standing in lines

Specific Situations

animals

heights

needles/blood

flying

thunderstorms

the dark

dentists

other (write-in)

other (write-in)

If you selected any of the above, how much does your fear or avoidance interfere with your normal daily life? (choose one)

no interference

mild/a little

moderate/medium interference

considerable/much interference

extreme/severe interference

In the preferred embodiment, Screen D—Social Phobia Screen will have the following introductory paragraph: “According to you previous responses, you have an excessive and unreasonable fear of or avoid social or work situations because you feel embarrassed, humiliated, or feel that people are judging you. Please answer the following questions about your experience.”

Also in the preferred embodiment, Screen D—Social Phobia Screen will include the following questions. The subject is asked to respond “yes” or “no”, unless multiple possible subsymptom answers are provided, in which cases the subject is asked to select all applicable answers.

Do you feel extremely awkward in social situations?

Which situations do you fear or avoid because you fear embarrassment, humiliation, or judgment?

speaking or performing in front of others

dealing with people in authority

talking to strangers

being observed (watched) while eating or drinking

social gatherings/parties

dating situations

being observed (watched) while working

being the center of attention

eye contact

speaking to people who you find attractive

How much does your fear or avoidance of the above situations interfere with your normal daily life?

no interference

mild/some interference

moderate/medium interference

considerable/much interference

extreme/severe interference

In the preferred embodiment, Screen E—OCD Screen will have three possible introductory paragraphs. The first reads: “According to your previous responses, you experience anxiety because of thoughts, images, or impulses you can't control. You also do certain things or repeat certain thoughts over and over again. You do these things according to special rules, or until it feels just right. Please answer these additional questions about your experience.”

The second reads: “According to your previous responses, you experience anxiety because of thoughts, images, or impulses you can't control. Please answer these additional questions about your experience.”

The third reads: “According to your previous responses, you do certain things or repeat certain thoughts over and over again. You do these things according to special rules, or until it feels just right. Please answer the following additional questions about your experience.”

In the preferred embodiment, Screen E—OCD Screen includes the following questions. The subject is asked to respond “yes” or “no”, unless multiple possible subsymptom answers are provided, in which case the subject is asked to select all applicable answers.

Do these thoughts, images, or impulses seem intrusive, strange, or inappropriate?

Do you try to resist, ignore, or suppress these thoughts, images, or impulses?

Are you able to resist, ignore, or suppress these thoughts, images, or impulses?

What are the general themes of your thoughts, images or impulses? (select the item for more details)

harm to self or others

sex

contamination, germs, disease

religion or blasphemy

doubting (second-guessing)

making decisions

counting, praying, repeating

checking

How much do these thoughts, images, or impulses interfere with your normal daily life? (choose one)

no interference

mild/a little interference

moderate/medium interference

considerable/much interference

extreme/severe interference

Check what you do over and over or according to special rules or until it feels “just right” (select the item for more details):

washing, cleaning

checking (locks, doors, stove . . . )

arranging/ordering/sorting/list making

repeating (words, numbers, prayers, movements . . . )

saving/collecting/hoarding

How much do the above behaviours interfere with your normal daily life? (choose one)

no interference

mild/a little interference

moderate/medium interference

considerable/much interference

extreme/severe interference

In the preferred embodiment, the questions relating to uncontrollable thoughts, images and impulses (the first 5 questions of Screen E—OCD Screen) will only be presented to the subject if he indicated in the pre-screen the presence of such uncontrollable thoughts, images and impulses. Similarly, the questions relating to repeating behaviours over and over (the last two questions of Screen E—OCD Screen) will only be presented to the subject if he indicated the presence of such behaviours in the pre-screen. If both categories of symptoms were indicated in the pre-screen, all of the questions of Screen E OCD Screen are presented.

In the preferred embodiment, Screen F—GAD Screen will include the following introductory paragraph: “According to your previous responses, for the past six months or more you have been worrying constantly or excessively about several different things (for example: work, school, family, finances, or health). Please answer the following questions about your worrying.”

Also in the preferred embodiment, Screen F—GAD Screen will include the following questions. The subject is asked to answer “yes” or “no”, unless multiple possible subsymptom answers are provided, in which case the subject is asked to select all applicable answers.

Do you worry much more than most people about things such as work, school, family finances, or health?

Do you worry about these things most of the time on most days?

Do you have difficulty controlling these worries, or do they interfere with your ability to concentrate and get things done?

Most of the time, when you're anxious or worried do you (check all that apply):

feel restless, keyed up, or on edge

feel tense

feel tired, weak, or easily exhausted

feel irritable

have difficulty concentrating, or find your mind going blank

have difficulty sleeping

How much does your worrying interfere with your normal daily life?

no interference

mild/a little interference

moderate/medium interference

considerable/much interference

extreme/severe interference

In the preferred embodiment, Screen G—PTSD Screen will include the following introductory paragraph: “According to your previous responses, you have experienced, witnessed, or been involved in a traumatic or life threatening event that included death or serious harm or the threat of death or serious harm to you or someone else, such as sexual assault, rape, accident, assault, disaster, war, or torture. Please answer the following questions about your experience of the event.”

Also in the preferred embodiment, Screen G—PTSD Screen will include the following questions. The subject is asked to answer “yes” or “no”. When multiple possible subsymptom answers are provided, the subject is asked to select all applicable answers.

During the event did you feel afraid, hopeless, helpless, terrified, or horrified?

Since the traumatic event have you: (please check the applicable answers)

re-experienced the event in disturbing ways such as dreams, memories, flashbacks, or physical reactions.

avoided thoughts, feelings, or conversations about the event.

avoided activities, places, or people that remind you of the event.

had difficulty remembering some important things about the traumatic event.

been less interested in your normal work, hobbies, or social activities.

felt detached, apart, or estranged from others.

felt emotionally numb or less able to have feelings.

felt that your life will be shorter because of the traumatic event.

had difficulty sleeping.

been especially irritable or had angry outbursts.

had difficulty concentrating.

felt constantly keyed up, nervous, or “on guard”.

been easily startled.

How long have you been experiencing these symptoms (choose one)?

less than 1 month

more than 1 month, less than 1 year

more than 1 year, less than 2 years

more than 2 years, less than 5 years

more than 5 years

How much does your experience of the traumatic event interfere with your normal daily life?

no interference

mild/a little interference

moderate/medium interference

considerable/much interference

extreme/severe interference

In the preferred embodiment, Screen H—Depression Disorder Screen has seven possible introductory paragraphs. One of the seven is displayed, depending upon what combinations of questions 9-11 of the pre-screen the subject answered “yes” to. The first introductory paragraph reads: “According to your previous responses, you have been feeling depressed, sad, or flat for most of the time for the past two weeks or more. Please answer the following questions about your experience.”

The second reads: “According to your previous responses, for the past two weeks or more you have lost interest or pleasure in the things usually like. Please answer the following questions about your experience.”

The third reads: “According to your previous responses, you have felt depressed, sad, or flat for most of the time for the last two years or more. Please answer the following questions about your experience.”

The fourth reads: “According to your previous responses, you have been feeling depressed, sad, or flat for most of the time for the past two weeks or more. You have lost interest or pleasure in the things that you usually like. You have also had a chronically depressed mood for most of the time for the past two years or more. Please answer the following questions about your experience.”

The fifth reads: “According to your previous responses, you have been feeling depressed, sad, or flat for most of the time for the past two weeks or more. You have also felt depressed, sad, or flat for most of the time for the last two years or more. Please answer the following questions about your experience.”

The sixth reads: “According to your previous responses, for the past two weeks or more have you lost interest or pleasure in the things that you usually like. You have also felt depressed, sad, or flat for most of the time for the last two years or more. Please answer the following questions about your experience.”

The seventh reads: “According to your previous responses, you have been feeling depressed, sad, or flat for most of the time for the past two weeks or more. You have also lost interest or pleasure in the things you usually like. Please answer the following questions about your experience.”

In the preferred embodiment, Screen H—Depression Disorder Screen includes the following questions. The subject is required to answer “yes” or “no”, unless multiple possible subsymptom answers are provided, in which case the subject selects all applicable answers.

Have you experienced constant low mood and irritability for the past two weeks or more?

For the past two weeks or more have you been: (choose all applicable answers)

gaining or losing weight

sleeping more or less

talking or moving more slowly

feeling tired or like you have no energy

having difficulty making decisions or concentrating

feeling guilty or worthless

thinking a lot about hurting yourself

wishing you were dead or feeling suicidal

How much does your depression or your loss of usual pleasures or interests interfere with your normal daily life?

no interference

mild/a little interference

moderate/medium interference

considerable/much interference

extreme/severe interference

When the subject completes the pre-screen, he submits the answers and they are received by the host computer. Similarly, the subject submits the answers to each of the detailed screens that he completes, and the answers are received by the host computer. An algorithm is applied by the software on the host computer. Preferably, the algorithm will determine the anxiety disorder, the precise anxiety disorder subtypes, and common comorbid conditions that correspond to the answers received by the host computer from the subject. These answers indicate the anxiety disorder symptoms, the anxiety disorder subsymptoms, the common comorbid condition symptoms and the common comorbid condition subsymptoms endorsed (and unendorsed) by the subject.

It will be appreciated that the questions contained in the screens are designed to elicit responses from the subject that will permit a doctor to diagnose anxiety disorders and their subtypes, as well as common comorbid conditions and their subtypes (e.g. depression and dysthemia), based on DSMIV criteria and other scientifically valid criteria. Thus the algorithm can determine which disorders, subtypes orcomorbid conditions may be present by comparing the answers received to a predetermined set of criteria stored within the host computer.

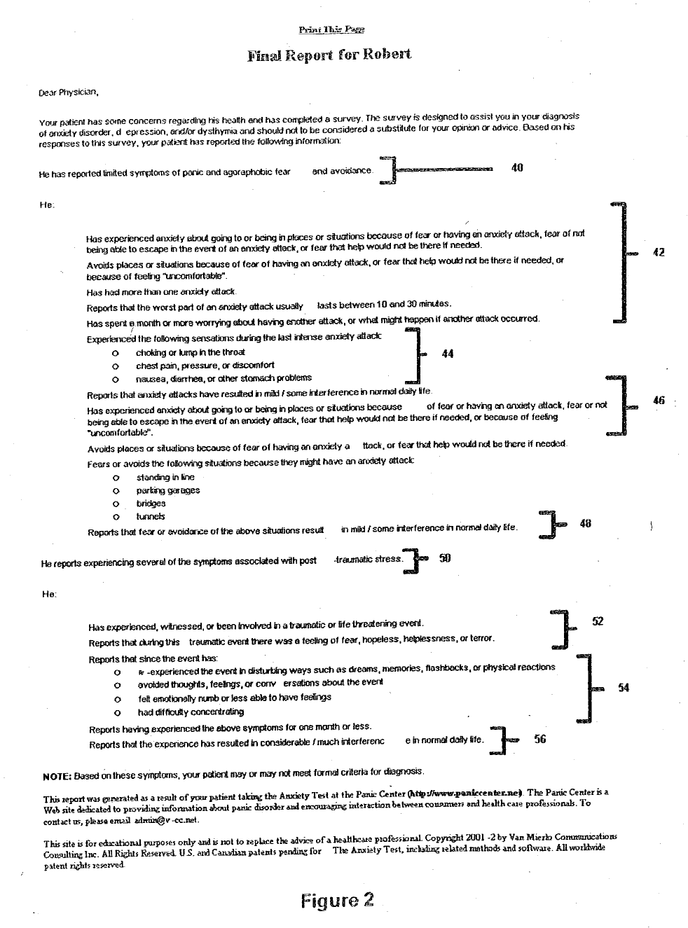

Most preferably, the host computer then generates a final report. A screen shot of a sample final report is shown at FIG. 2. Preferably the final report identifies the first name of the subject, which was preferably requested from the subject at the outset of the test. In FIG. 2 at the top, it is stated that FIG. 2 is a “Final Report for Robert”. It will be appreciated that it is preferable to use first names only, to preserve anonymity in case the report falls into the hands of a third party.

As shown in FIG. 2, the final report also preferably indicates the breadth of the test. Thus, in the first paragraph, the final report of FIG. 2 indicates that the subject was screened for anxiety disorder, depression and dysthymia. Together with the summary of symptoms and subsymptoms below, this indicates to the doctor what symptoms and subsymptoms were not endorsed. This is because the doctor knows that, since the subject was tested for anxiety disorders, depression and dysthymia, if any of those conditions are not shown on the final report, then the subject did not endorse symptoms indicating their presence. It will be appreciated that there are other ways to indicate unendorsed symptoms and subsymptoms (e.g. by listing them), and these are comprehended by the invention. However, the method described above is preferred because it provides the necessary information to the doctor while allowing the final report to be relatively short and easy to read. By contrast, listing all unendorsed symptoms would render the final report longer and more difficult to read.

As shown in sections 40 and 50 of FIG. 2, the final report preferably describes the anxiety disorder and/or common co-morbid condition, as well as the precise subtype, corresponding to the subject's answers. Thus, for example at section 40, the subject is said to have reported “limited symptoms of panic”. This description indicates that the broad anxiety disorder category referred to is panic disorder, and the subtype is “limited symptoms”. In this case, as with some of the other subtypes listed below, a subtype is identified which does not fit the formal criteria for the main disorder type, but which would be important for a doctor to know. Thus, although Robert has not reported symptoms of full-blown panic disorder, he does have limited symptoms of panic. This would be important for a doctor to know in helping the subject. Similarly, the report of FIG. 2 reports “agoraphobic fear and avoidance”, two subtypes of agoraphobia. By using the term “agoraphobic”, the report indicates to the doctor that it is identifying agoraphobia as the disorder type being discussed. “Agoraphobic fear and avoidance” are two subtypes which do not rise to the level of agoraphobia proper, but which a doctor should be aware of in helping the patient.

At section 50, the report also indicates that the subject reported “symptoms of post-traumatic stress”. Thus, in this case, PTSD is the disorder, and “several symptoms” is the subtype. Although this doesn't rise to the level of PTSD, it is important for the doctor to know of the symptoms.

It will be appreciated that “subtype” does not only include subtypes that do not rise to the precise DSM-IV definition of the disorder or condition. It also includes subtypes within the DSM-IV definitions. For example, the host computer and algorithm are preferably programmed to be able to identify Social Phobia Non-Generalized Subtype (public speaking) and Social Phobia Generalized Subtype, which both constitute social phobia. It will be appreciated that the purpose of identifying subtypes is to provide the doctor with more precise information to assist in diagnosis.

Sections 44 and 54 show summaries of endorsed subsymptoms. “Subsymptoms” as used in this specification refers to specific manifestations of symptoms. Thus, for example, at section 44, 3 subsymptoms of Robert's anxiety attacks are shown. The presence of anxiety attacks themselves is a symptom, as is the fact that Robert experienced some physical or emotional difficulties as part of the attack. However, the specific difficulties he experienced are subsymptoms. The 5^{th} question of Screen A list 12 different possible subsymptoms, of which Robert endorsed three.

Similarly, each detailed screen in the preferred embodiment contains at least one question which requires the subject to select one or more subsymptoms. For example, in the agoraphobia screen, the subject is asked which situations he fears or avoids, and is given a long list of subsymptoms. It will be appreciated that the purpose of summarizing subsymptoms is to provide the doctor with as much diagnostic information as possible. In some cases, the doctor's diagnosis or treatment may change depending on which subsymptoms are endorsed.

The final report is preferably an original final report as shown in FIG. 2. The final report of FIG. 2 is original in the sense that it is not merely a printout of the questions and answers given by the subject. Rather, it provides a reformatted summary that does not require the doctor to re-read the test just taken by the subject. Instead, the original final report summarize the endorsed and unendorsed symptoms directly.

As shown in sections 48, 50, the final report preferably reports the extent to which reported symptoms and subsymptoms affect the subject's life. This is based on questions to that effect in the detailed screens. It will be appreciated that the degree that the subject's life is affected is often a relevant DSM-IV criterion. Furthermore, this information is useful for both doctor and subject in helping them decide what approach to take to dealing with the subject's problems. The more a problem interferes with a subject's life, the more effort he may be willing to make for treatment.

In the preferred embodiment, the software can group symptoms under 25 different headings for final reports, each heading representing an anxiety disorder and/or common co-morbid condition, as well as a subtype if applicable. These headings are (1) Acute Stress Disorder, (2) Agoraphobic Anxiety, (3) Agoraphobic Avoidance, (4) Agoraphobia without a history of Panic Disorder, (5) Dysthymic Disorder, (6) Dysthymic Disorder (chronic depressive symptoms), (7) GAD, (8) Limited Symptom Panic Disorder with Agoraphobia, (9) Limited Symptom Panic Disorder with Agoraphobic Avoidance, (10) Limited Symptom Panic Disorder with Agoraphobic Fear, (11) Major Depressive Disorder, (12) Major Depressive Disorder (depressive symptoms), (13) Obsessive-Compulsive Disorder, (14) Obsessive-Compulsive Disorder Compulsive Symptoms, (15) Obsessive-Compulsive Disorder Primary Obsessional, (16) Panic and Agoraphobic Fear, (17) Panic Disorder with Agoraphobia, (18) Panic Disorder with Agoraphobic Avoidance, (19) Panic Disorder without Agoraphobia, (20) Panic Disorder without Agoraphobia with Limited Symptom Panic Attacks, (21) Social Phobia Non-Generalized Subtype (public speaking), (22) Social Phobia Generalized Subtype, (23) Specific Phobia, (24) PTSD and (25) PTSD Symptoms.

It will be appreciated that, for the most part, the headings described above represent possible diagnoses for anxiety disorders (and applicable subtypes, if any), or for common co-morbid conditions (e.g. Major Depressive Disorder) as defined in the DSM-IV. However, as described above, some of the headings do not represent full-blown disorders or other conditions as defined in the DSM-IV. Rather, these other headings are used to identify disorders or conditions when the subject has reported related symptoms which do not meet the formal definitions of the DSM-IV, but which are still deemed sufficiently important that the subject should consult a physician. Thus, for example, if the anxiety test discloses that the subject fears subways and elevators because he fears he will have an anxiety attack and that help will not be available, but does not report that he avoids these places, this would not be sufficient for a diagnosis of agoraphobia according to formal DSM-IV criteria. However, it is preferable that the subject discuss these symptoms with his physician regardless. There are two reasons for this. First, the subject may have agoraphobia, but may for some reason have failed to report all the symptoms. This is more likely to be discovered if a physician makes further inquiries directly with the subject. Second, even though the formal criteria for the disorder have not been met, the subject may wish to receive treatment or therapy for his condition anyway. Thus, in the example just described, the software will preferably produce a final report summarizing the reported symptoms under the heading of “Agoraphobic Anxiety”. This indicates to the physician that the patient is experiencing anxiety symptoms of an agoraphobic nature.

It will be appreciated by those skilled in the art that this preferred method of reporting provides useful diagnostic information to a doctor. By reporting to the doctor a summary of the symptoms and subsymptoms reported by the subject, significant time is saved for the doctor. This is because, in order to make a diagnosis of anxiety disorders and/or common co-morbid conditions, the doctor would need to ask the questions that have been asked in the anxiety test. Then, using the answers provided by the subject, the doctor may diagnose one or more disorders. Thus, if the doctor were to receive a report which simply indicates the possibility that an anxiety disorder is present, or simply indicates the possibility of a specific anxiety disorder, the doctor would still have to make his own inquiries to satisfy himself as to the presence of symptoms. By providing summaries of symptoms and subsymptoms, the need for the doctor to repeat the same questions is obviated. Instead, the doctor can ask any more detailed follow-up questions that he believes are necessary in the circumstances, and proceed to make a diagnosis.

As a result, it will be appreciated that this preferred format for the final report is particularly useful for primary care physicians. As described above, primary care physicians typically see a significant number of patients per day, and are unable to spend large amounts of time with any one patient. Thus, it is often difficult for primary care physicians to find the time to ask detailed questions about the symptoms of a wide variety of anxiety disorders. However, through use of the preferred anxiety test, the subject can answer, in advance of seeing the doctor, the necessary questions relating to a wide variety of anxiety disorders and co-morbid conditions, and can also provide his doctor with a report that summarizes the symptoms and subsymptoms that the subject has. As such, the doctor is not required to spend a large amount of time determining symptoms, but may instead proceed directly to follow-up questions and diagnosis.

Preferably, the software will provide the subject with functionality that facilitates the forwarding of the final report to the subject's doctor. Most preferably, this includes providing a printer-friendly version of the final report, thus making it easier for the subject to print out the final report and take it to his doctor. Also, most preferably, the software will facilitate the subject e-mailing the final report directly to his doctor when the final report is generated. This is preferably accomplished by providing a button on the screen which calls the subject's email programs and provides a new message, attaching the final report to be sent to the doctor.

In the preferred embodiment, as a population of subjects is tested, the software keeps data comprising at least the following statistics: totals of each answer (symptoms and subsymptoms) endorsed and unendorsed; combinations of answers (symptoms and subsymptoms) endorsed and unendorsed; anxiety disorders and/or common co-morbid conditions, as well as subtypes, determined for the subjects within the population; and final reports generated for subjects within the population. Also, preferably, the subject will be questioned for his or her sex before the test, and the data will include the sex of the subjects. Also, the other data described above will preferably be categorized and made available by sex.

Preferably, this data will be made available to researchers so as to permit the data to be used for research relating to anxiety disorders. It will be appreciated that, if the anxiety test is implemented, as preferred, via at least one computer, the software can efficiently collect data on all of the answers given and not given by subjects taking the test. Thus, when access to the data is provided to researchers, they can obtain the data in the form of data units, i.e. units of data which indicate either the prevalence or non-prevalence of a certain symptom or diagnosis or other relevant unit of information within the test. Most preferably, this collection of data is implemented by assigning a unique code to each question and each possible answer in the anxiety test.

It will be appreciated that each piece of text used in the final reports to describe a reported symptom or subsymptoms matches a question asked in either the pre-screen or the detailed screens of the anxiety test. Thus, for example, if the subject answered “yes” to the question: “Have you had more than one anxiety attack?”, there will be a corresponding pre-determined piece of text used in the final report to summarize this symptom, which preferably is, “has had more than one anxiety attack.” Most preferably, a unique code is also assigned to each of these pieces of text used in the final reports to describe the specific symptoms reported by the subject. It is also most preferable that a unique code be assigned to each disorder/condition description or heading (e.g. “Panic Disorder with Agoraphobia”; “Agoraphobic Anxiety”) used in final reports to categorize symptoms reported by the subject.

It will be appreciated that the software can efficiently keep data on the various questions asked (and not asked) and answers given (and not given) by the population of subjects that takes the anxiety test. Since the test is computerized, the software can simply save in a database the codes of questions asked, answers given and not given, each symptom and subsymptom description summarized in the final reports, and each disorder and/or subtype description used in the final reports. To facilitate access to the data by researchers, they can be given a database map which shows what unique code applies to each question, answer, symptom and subsymptom description and disorder description. Preferably, the access to the data is provided over the Internet. Thus, researchers from all over the world can have access to the data, at any time of the day or night, everyday. However, it will be appreciated that the researchers seeking access to the data are preferably required to register and be issued a password, which they will have to use to gain access to the data.

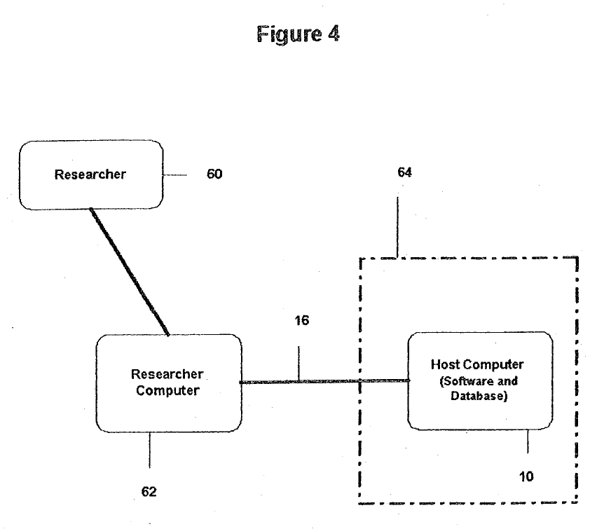

FIG. 4 shows a preferred mode of data access for the researcher 60. Through a research computer 62 having an Internet connection 16, the researcher can search the database in the host computer 10. Preferably, the host computer 10 is protected by a security firewall 64 to prevent unauthorized access to the host computer.

It will be appreciated that this preferred form of the anxiety test can provide a wide variety of useful data for researchers seeking to do research into anxiety disorders. There are a number of reasons for this. First, because the anxiety test is preferably offered via the Internet, a very large population of potential subjects has access to the test. Subjects are not constrained by location, and can take the test from anywhere in the world. Thus, it is possible to obtain large volumes of data on the population relatively cheaply. This is to be contrasted with paper and pencil instruments, where it would likely be much more expensive to obtain similar volumes of data.

Second, the anxiety test asks questions relating to a broad range of disorders and conditions. Thus, not only can data relating to specific symptoms and specific anxiety disorders be collected, but data showing correlations between different disorders, conditions, symptoms and subsymptoms can be collected. This includes highly specific combinations which could not be explored using data from a narrower test. For example, using the anxiety test of the present invention, researchers can determine what proportion of people having the symptoms of Social Phobia Non-Generalized Subtype completed the OCD Screen and did not qualify as having OCD, but still indicated a compulsive urge for checking. As can be seen from this example, and from the wide variety of questions in the anxiety test as described above, the anxiety test can be used to provide large volumes of data in respect of diverse disorders, conditions, symptoms, and subsymptoms and combinations thereof.

The software will preferably be programmed to record the dates on which subjects take the anxiety test. Therefore, if desired, researchers will be able to limit their inquiries to certain date ranges.

Preferably data will also be kept in the form of statistics on what country or geographical location each subject in the population is located in. The other statistics are preferably made available in combination with the country information. It will be appreciated that this can also provide very useful information to researchers. For example, through such information, researchers may be able to determine that certain disorders manifest themselves slightly differently, on average, in different countries. So, for example, the data may show that Chinese people who are depressed manifest it most commonly by sleeping too much, while French people who are depressed manifest it by eating too much. If such differences appear in the data, that information could possibly be used to improve treatments in specific geographical areas.

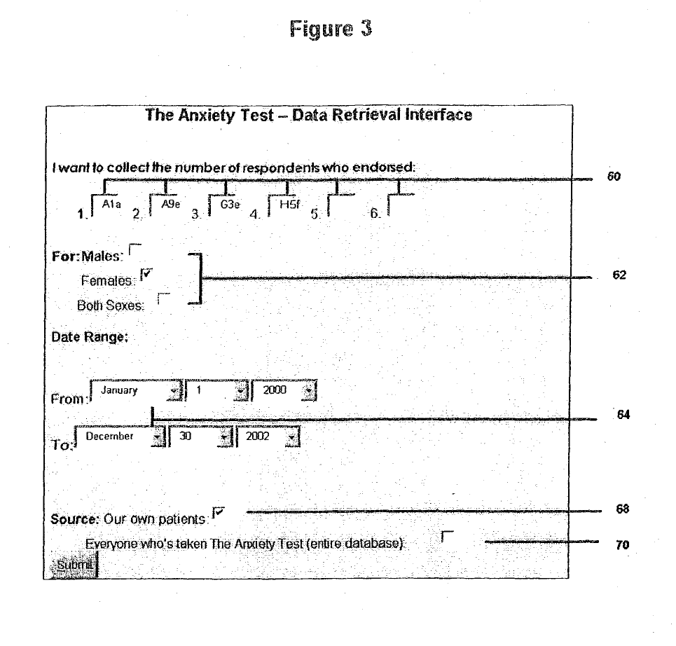

FIG. 3 shows an example of a software interface on the host computer through which researchers can search for and get data from the database. The boxes 60 allow the researchers to enter the codes for desired combinations of statistics, such as answers, symptoms and subsymptoms (endorsed and unendorsed) final reports, disorders, conditions and subtypes. The boxes 62 allows the search to be restricted by sex. The date range boxes 64 allow the search to be restricted by sex. The country box 66 preferably allows a country to be selected (or all countries).

The interface in FIG. 3 also allows individual research groups, registered on the host computer, to search through the whole population, or to restrict the search to subjects affiliated with the research group (boxes 68, 70). Thus, the software will preferably record and keep statistics on the affiliation of each subject, if any. It will be appreciated that the subjects need not be affiliated with any particular research group, but will often be members of the general public.

It will be appreciated that, in addition to facilitating research, the data can be used to modify the anxiety test itself in order to improve the test and in order to improve the collection of data. Thus, preferably, the software will be programmable so that an alert is issued (preferably electronically) when a previously specified data threshold is reached. That threshold could be, for example, that a certain absolute number or a certain percentage of subjects give a certain answer to a certain question in the anxiety test. In turn, using the alert to determine that the threshold has been reached, the test can be modified in response to this information. Thus, for example, if a high proportion of subjects that take the test (say 75%) are answering “yes” to a particular question within one of the detailed screens, it may be worthwhile to modify the screeners by placing that question in the pre-screen in order to make the anxiety test more efficient.

It will be appreciated that in the preferred embodiment the threshold can be set in respect of virtually any aspect of the test, including but not limited to the screens presented, answers given and not given, number of people taking the test, final reports (or their components) used or not used, and any combination thereof. Thus, to give another example, if a specified number of people take the test without ever endorsing a certain answer or being presented with a certain screen, the answer or screen may be removed or modified.

It will also be appreciated that modification in response to a threshold need not be done via an alert. For example, the software is preferably programmable as to be able to automatically modify the test when a threshold is reached, if such automatic modification is desired.

Those skilled in the art will appreciate that this method of modification allows the test to learn, grow, and adapt to its environment. By modifying the test in response to thresholds, the test can have form and content best suited to the data and the population, and can change for the better as circumstances, such as the data and the population, change.

This feature can also be used to facilitate better data collection. Thus, for example, if a large proportion of subjects are answering a particular question in a particular way, this may indicate the possibility of obtaining additional useful data about the relevant symptom. Thus, in response, the test could be modified to add extra follow-up questions which seek more specific information from the subject about the particular symptom. Similarly, if the data show that virtually no subjects say “yes” to a particular question, that question could be removed altogether. It will be appreciated, then, that the data can be used to modify the anxiety test, both by relocating questions within the test and by adding or removing questions.

As another example, the data can preferably be categorized by date, and the software programmed, to issue a notification if certain answers are given more often in certain date ranges. Thus, for example, if depressive symptoms ate being indicated significantly more often during winter months, then the test could be modified in response to add questions for the purpose of obtaining data on Seasonal Affective Disorder.

Thus, it will be appreciated that the test can be modified in a number of ways in response to a threshold being reached, including modifying the questioning process, adding, deleting or changing one or more screeners, adding, deleting or changing questions in the screeners, adding or subtracting disorders and conditions screened for, modifying the final reports (including information, contents, formatting and text) and combining question sets and/or screeners.

The software is preferably able to issue alerts not only for modification, but also for researchers. Thus, the software preferably can issue alerts to researchers when a threshold has been reached. This way, a researcher who wants to know for research purposes if a certain data threshold has been reached does not need to keep checking repeatedly. Instead, the software is programmed to send him on alert when the threshold is reached.

Preferably, the software will also be programmed to record which data units are requested by researchers, and which are not. Thus, for example, these records may show that researchers are closely following the co-morbidity of two particular anxiety disorders, but not the co-morbidity of others. Or, they may show that researchers are studying correlations between certain symptoms, but not others. Or, they may show that researchers are following the prevalence of certain disorders without regard to sex, and are thus not inquiring into how sex differences affect the prevalence of these disorders. In this way, the software can keep statistics showing what combinations of data units are being used by researchers and which are not. Preferably, these statistics will be kept not only for all researchers as a group, but for each individual researcher that is registered to use the data.

Preferably, full records are kept in respect of the data units requested by researchers. Thus, the software preferably records at least the following: (1) which data units are requested (this refers to all data units kept for the population of subjects); (2) the combinations of data units that are requested; (3) the identity of the researchers making each request; (4) the frequency that each data unit is requested; and (5) combinations of all of the above.

It will be appreciated that such records can be employed in a number of beneficial ways. First, the presence of such records allows for superior coordination of research projects among different researchers, even when the researchers are not actively cooperating. This is because the records can show what research is not currently being done and what inquiries are not being made by identifying what data units are frequently and infrequently requested. Thus, they can be used to reduce the probability of repetitive research by providing information to help researchers determine what research is not being done.

Second, the presence of such records allows for a more thorough evaluation of the research being done by researchers who use the data.

Specifically, a reviewer of the research can use the records to determine whether there are combinations of data units that would have been relevant to the issue being researched, but that were not used by the researcher. This also provides a way in which research projects can be evaluated on an ongoing basis to ensure that the specific research topic is being covered comprehensively.

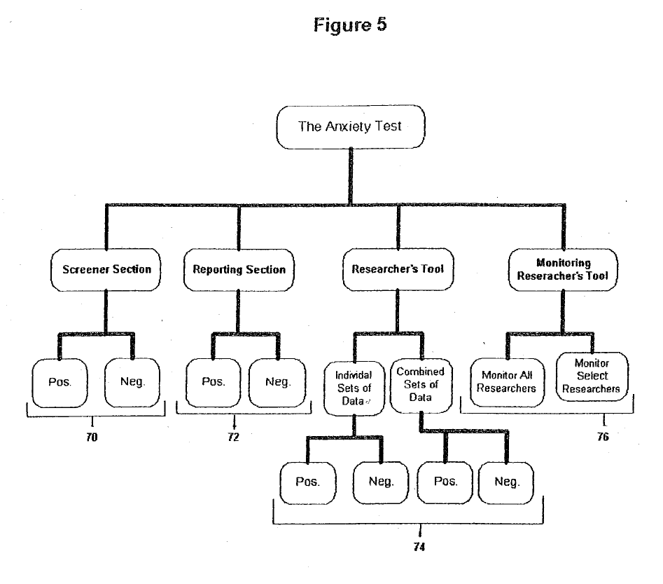

FIG. 5 is a chart showing the preferred primary functions of the software, including anxiety testing 70, reporting 72, researcher data access 74, and researcher data request monitoring 76.

It will be appreciated that all of the methods described above can be performed in relation to one or more of any medical or psychological conditions.

The testing need not be confined to anxiety disorders and co-morbid conditions, but can relate to testing the subjects' experience of one or more medical or psychological conditions.

It will be appreciated that while the present invention has been described in the context of various methods including methods for obtaining information, collecting data, modifying a system, notifying in respect of a threshold, the system, processes and methods of the present invention are capable of being distributed in a computer program product comprising a computer readable medium that bears computer usable instructions for one or more processors.

The medium may be provided in various forms, including one or more diskettes, compact disks, tapes, chips, wireline transmissions, satellite transmissions, Internet transmissions or downloadings, magnetic and electronic storage media, digital and analog signals, and the like. The computer usable instructions may also be in various forms, including compiled and non-compiled code.

Embodiments of and modifications to the described invention that would be obvious to those skilled in the art are intended to be covered by the appended claims. Some variations have been discussed above, and others will be apparent. For example, though use of the internet is preferred for diagnostic information and data collection is preferred, it is not required. Thus, for example, this invention could be used within a research group on a local area network, or could be used without any network at all.

Also, though the test can be taken by subjects in the privacy of their homes as described above, the test may also be taken by subjects in clinics, hospitals and other health care facilities, where a diagnosing physician is available on the spot. The test may also be taken by the subject from any other location.

The Embodiments of the Invention in Which an Exclusive Property or privilege is claimed are defined as follows:

1. A method of obtaining diagnostic information relating to anxiety disorders, the method comprising the steps of:
(1) asking a subject questions relating to anxiety disorder symptoms, via computer;
(2) receiving answers to the questions;
(3) based on the answers, generating via the computer an original final report;
(4) summarizing in the final report endorsed anxiety disorder symptoms;
(5) indicating in the final report unendorsed anxiety disorder symptoms.

2. The method of claim 1, wherein said method further comprises the step of asking the subject questions relating to common co-morbid condition symptoms, the method further comprising the steps of summarizing in the final report endorsed common co-morbid condition symptoms and of indicating in the final report unendorsed common co-morbid condition symptoms.

3. The method of claim 2, said method further including the steps of identifying in the final report anxiety disorders corresponding to the endorsed anxiety disorder symptoms, and identifying in the final report the precise subtypes of the anxiety disorders corresponding to the endorsed anxiety disorder symptoms.

4. The method of claim 3, wherein the method further comprises the step of asking the subject questions relating to anxiety disorder subsymptoms, and the step of summarizing in the final report endorsed anxiety disorder subsymptoms.

5. The method of claim 3, wherein the method includes the step of questioning a subject for at least his first name and for his sex, and the step of indicating at least the subject's first name on the final report.

6. The method of claim 1, wherein the method further includes the step of asking the subject the degree to which the endorsed anxiety disorder symptoms affect the subject, and the step of indicating on the final report the degree to which the endorsed anxiety disorder symptoms affect the subject.

7. The method of claim 4, the method further comprising the step of asking the subject the degree to which the endorsed anxiety disorder subsymptoms affect the subject, and the step of indicating on the final report the degree to which the endorsed anxiety disorder subsymptoms affect the subject.

8. The method of claim 2, the method further comprising the step of asking the subject the degree to which the endorsed common co-morbid condition symptoms affect the subject, and the step of indicating on the final report the extent to which the endorsed common co-morbid condition symptoms affect the subject.

9. The method of claim 2, wherein the method further comprises the step of asking the subject about common co-morbid condition subsymptoms and the step of summarizing in the final report endorsed common co-morbid condition subsymptoms.

10. The method of claim 9, wherein the method further comprises the step of asking the subject the degree to which the endorsed common co-morbid condition subsymptoms affect the subject, and the step of indicating on the final report the degree to which the endorsed common co-morbid condition subsymptoms affect the subject.

11. The method of claim 8, the method further comprising the step of generating a printer friendly version of the final report.

12. The method of claim 8, the method further comprising the step of facilitating the sending of the final report by electronic mail to a health care practitioner.

13. The method of claim 1, the asking step comprising the steps of (1) asking the subject questions via a pre-screen, and (2) asking the subject questions via detailed screens, wherein the identity of the detailed screens is determined from the answers received from the subject on the pre-screen.

14. A method of collecting data relating to anxiety disorders, the method comprising the steps of:
A) obtaining information from a population of subjects wherein each subject is questioned according to a subject interaction method comprising the steps of;
(1) questioning each subject via computer for anxiety disorder symptoms;
(2) receiving answers showing endorsed and unendorsed anxiety disorder symptoms;
B) keeping data comprising statistics on combinations of the answers given by the subject within the population, wherein it is possible to determine a number of subjects that gave particular combinations of answers.
C) making the data available to researchers

15. The method of claim 14, wherein the subject interaction method further comprises the step of determining, based on the answers, the anxiety disorders, and the precise subtypes of the anxiety disorders, corresponding to the endorsed anxiety disorder symptoms, the data further comprising statistics on the anxiety disorders and the precise subtypes determined for the subjects within the population.

16. The method of claim 14, wherein the subject interaction method further comprises the step of generating a final report summarizing the endorsed anxiety disorder symptoms and indicating anxiety disorders corresponding to the endorsed anxiety disorder symptoms, the data further comprising statistics on final reports generated for the subjects with the population.

17. The method of claim 14, the subject interaction method further comprising the steps of questioning each subject via computer for common comorbid condition symptoms and receiving answers showing endorsed and unendorsed common co-morbid condition symptoms within the population, the data further comprising statistics on endorsed and unendorsed common comorbid condition symptoms, and statistics on co-morbid condition symptoms for subjects within the population, wherein it is possible to determine a number of subjects that gave particular combinations of endorsed and unendorsed common co-morbid condition symptoms.

18. A method of collecting data relating to anxiety disorders, the method comprising the steps of:
A) obtaining information from a population of subjects by questioning each subject according to a subject interaction method comprising the steps of:
1) questioning each subject via computer for anxiety disorder symptoms and anxiety disorder subsymptoms;
2) receiving answers showing endorsed and unendorsed anxiety disorder symptoms and anxiety disorder subsymptoms;
B) keeping data comprising statistics on endorsed and unendorsed anxiety disorder symptoms within the population and endorsed and unendorsed anxiety disorder subsymptoms within the population.
C) making the data available to researchers.

19. The method of claim 18, wherein the data further comprises statistics on combinations of endorsed and unendorsed anxiety disorder symptoms and endorsed and unendorsed anxiety disorder subsymptoms wherein it is possible to determine a number of subjects that gave particular combinations of endorsed and unendorsed anxiety disorder symptoms and subsymptoms.

20. The method of claim 18, wherein the method further comprises the step of keeping track of the data each subject is questioned, the method further comprising the step of making the data available to researchers according to a selected data range.

21. The method of claim 18, the subject interaction method further comprises the steps of requesting the sex of each subject and the step of receiving the sex of each subject, the method further comprising the step of making the data available to researchers according to a selected sex.

22. A method of collecting information on research being conducted-, the method comprising:
(1) providing a collection of data units relating to one or more medical or psychological conditions;
(2) receiving requests for data units from one or more researchers;
(3) recording which data units are requested.

23. The method of claim 22, wherein the method further comprises the step of recording the frequency that requested data units are requested.

24. The method of claim 22, wherein the method further comprises the step of identifying unrequested and infrequently requested data units.

25. The method of claim 22, wherein the method further comprises, prior to the providing step, the step of collecting data units by questioning a population of subjects for information relating to each subject's experience with the one or more medical or psychological conditions.

26. The method of claim 23, wherein the providing step comprises providing a collection of data contained in a computer database, and the receiving step comprises receiving the requests via a computer.

27. The method of claim 26, wherein the providing step comprises providing a collection of data contained in a computer database connected to the Internet, and the receiving step comprises receiving the requests via the Internet.

28. The method of claim 26, wherein the method further comprises the step of recording combinations in which data units are requested by one or more researchers.

29. The method of claim 23, further comprising the step of recording an identity of each researcher and recording which data units are requested by each researcher.

30. A method of modifying a system for determining and reporting information relating to anxiety disorders, the method comprising:
(1) via a computer, questioning a population of subjects, according to a pre-existing questioning process, for information relating to anxiety disorders;
(2) via the computer, receiving answers from the subjects;
(3) determining from the answers if a previously specified data threshold has been reached;
(4) if the threshold has been reached, automatically and via the computer modifying the questioning process.

31. The method of claim 30, wherein the questioning step comprises questioning the population of subjects through a series of screeners, the modifying step comprising taking an action selected from adding at least one screener, deleting at least one screener, and changing at least one screener.

32. The method of claim 30, wherein the questioning step comprises questioning the population of subjects through at least one set of questions, the modifying steps comprising taking an action selected from adding to the at least one question set, deleting at least one question from the at least one question set, changing at least one question from the at least one question set, and reordering questions in the at least one question set.

33. The method of claim 30, wherein the questioning step comprises questioning on a pre-existing set of anxiety disorder symptoms, and wherein the modifying step comprises changing the pre-existing set of anxiety disorder symptoms.

34. The method of claim 30, the method further comprising the step of generating final reports, including previously specified aspects thereof, based on the answers given by the subjects, and wherein the method further comprises the step of, if the threshold had been reached automatically and via the computer modifying at least one of the previously specified aspects of the final reports.

35. The method of claim 34, wherein the previously specified aspects include the types of information shown on the final reports, and the order in which information is presented on the final reports, predetermined text used on the final reports, and the visual formatting of the final reports.

36. The method of claim 30, wherein the questioning step comprises the step of questioning the population of subjects through a preexisting plurality of question sets including first and second question sets, the modifying step comprising combining the first and second question sets.

37. A data threshold notification method, the method comprising the steps of:
1) via a computer, questioning a population of subjects for information relating to one or more medical or psychological conditions;
2) via the computer, receiving answers from the subjects;
3) determining from the answers if a previously specified data threshold had been reached;
4) if the threshold has been reached, automatically issuing an alert.

38. The method of claim 37, wherein the issuing step comprises issuing the alert electronically.
